(19) United States
(12) Patent Application Publication
(10) Pub. No.: US 2006/0257483 A1
(43) Pub. Date: Nov. 16, 2006

(54) Controlled release bupropion dosage forms

(75) Inventors: Tianrun Yang, Coral Springs, FL (US); Timothy Weng, Cooper City, FL (US)

Correspondence Address: DAVIDSON, DAVIDSON & KAPPEL, LLC, 485 SEVENTH AVENUE, 14TH FLOOR, NEW YORK, NY, 10018, US

(73) Assignee: ABRIKA Pharmaceuticals, Sunrise, FL (US)

(21) Appl. No.: 11/125,857

(22) Filed: May 10, 2005

(51) Int. Cl. A61K 9/24; A61K 31/195

(52) U.S. Cl. 424/471; 514/563

(57) ABSTRACT

A controlled release pharmaceutical dosage form comprising bupropion or pharmaceutically acceptable salt thereof.

### FIELD OF THE INVENTION

The present invention is directed to controlled release bupropion dosage forms which in certain embodiments are suitable for once-a-day administration.

### BACKGROUND OF THE INVENTION

Bupropion is a widely used antidepressant drug and is commercially available in the hydrochloride salt form under the tradename Wellbutrin® by GlaxoSmithKline. This product is available in 75 mg and 100 mg immediate release tablets. It is known in the art that bupropion hydrochloride may induce certain side effects. For example, seizures can occur in about 0.4% patients and it has been reported that this effect is due to the high peak plasma concentrations of bupropion provided by immediate release formulations. The development of extended controlled release dosage forms has been considered as an appropriate means to overcome this situation.

Wellbutrin SR® and Wellbutrin XL®, commercially available by GlaxoSmithKline, are two currently marketed bupropion hydrochloride controlled release dosage forms.

Wellbutrin SR® formulations are supplied for oral administration as 100 mg, 150 mg and 200 mg, film-coated, extended-release tablets suitable for twice-a-day administration (e.g., the usual adult target dose for Wellbutrin SR® Tablets is 300 mg/day, given as 150 mg twice daily). Each Wellbutrin SR® tablet contains the labeled amount of bupropion hydrochloride and the inactive ingredients: carnauba wax, cysteine hydrochloride, hydroxypropyl methylcellulose, magnesium stearate, microcrystalline cellulose, polyethylene glycol, polysorbate 80, and titanium dioxide, along with the respective colorants for each strength.

Wellbutrin XL® formulations are supplied for oral administration as 150 mg and 300 mg creamy-white to pale yellow extended release tablets suitable for once-a-day administration (e.g., the usual adult target dose for Wellbutrin XL® tablets is 300 mg/day given once daily in the morning). Each Wellbutrin XL® tablet contains the labeled amount of bupropion hydrochloride and the inactive ingredients: ethylcellulose aqueous dispersion (NF), glyceryl behenate, methacrylic acid copolymer dispersion (NF), polyvinyl alcohol, polyethylene glycol, povidone, silicon dioxide, and triethyl citrate.

The FDA Publication Entitled “Approved Drugs Products with Therapeutic Equivalence,” commonly referred to as the “Orange Book” lists U.S. Pat. Nos. 6,096,341 and 6,143,327 as purportedly encompassing Wellbutrin XL® tablets.

### OBJECTS AND SUMMARY OF THE INVENTION

It is an object of the present invention to provide a controlled release oral dosage form comprising bupropion or a pharmaceutically acceptable salt thereof.

It is a further object of certain embodiments of the present invention to provide a method for preparing a controlled release dosage form comprising bupropion or a pharmaceutically acceptable salt thereof.

It is a further object of certain embodiments of the present invention to provide a method of treating depression by administering the controlled release dosage forms disclosed herein comprising bupropion or a pharmaceutically acceptable salt thereof to a human patient in need of such treatment.

It is a further object of certain embodiments of the present invention to provide a controlled release dosage form comprising bupropion or a pharmaceutically acceptable salt thereof which provides a therapeutic effect such that the dosage form is administrable to a subject on a once-daily basis.

It is a further object of certain embodiments of the present invention to provide a controlled release dosage form comprising bupropion or a pharmaceutically acceptable salt thereof, which provides therapeutically effective blood levels of the bupropion for at least 24 hours at steady state.

It is a further object of certain embodiments of the present invention to provide a controlled release dosage form comprising bupropion or a pharmaceutically acceptable salt thereof which is bioequivalent to Wellbutrin XL®.

The above-mentioned objects and others are achieved by virtue of the present invention, which is directed in part to a controlled release oral solid dosage form comprising a controlled release core comprising bupropion or a pharmaceutically acceptable salt thereof and a controlled release material; the core overcoated with an enteric coating.

In certain embodiments, the core comprises a controlled release matrix comprising the bupropion or pharmaceutically acceptable salt thereof dispersed in the controlled release material. In certain embodiments, the controlled release matrix is a compressed tablet.

In certain embodiments, the present invention is directed to an oral solid dosage form comprising a therapeutically effective amount of bupropion or a pharmaceutically acceptable salt thereof and one or more pharmaceutically acceptable excipients, the dosage form providing an in-vitro dissolution rate using USP Apparatus I (baskets) at 75 rpm in 1000 mL of 0.1N. HCl of not more than about 10% of bupropion or pharmaceutically acceptable salt thereof released after a time point selected from the group consisting of 2 hours, 4 hours, 8 hours, 12 hours, or 16 hours, the formulation providing a therapeutic effect such that the dosage form is administrable to a subject on a once-daily basis.

In certain embodiments, the present invention is directed to an oral solid dosage form comprising a therapeutically effective amount of bupropion or a pharmaceutically acceptable salt thereof and one or more pharmaceutically acceptable excipients, the dosage form providing an in-vitro dissolution rate using USP Apparatus I (baskets) at 75 rpm in 1000 mL of 0.1N. HCl of not more than about 5% of bupropion or pharmaceutically acceptable salt thereof released after a time point selected from the group consisting of 2 hours, 4 hours, 8 hours, 12 hours, or 16 hours, the formulation providing a therapeutic effect such that the dosage form is administrable to a subject on a once-daily basis.

In certain embodiments, the present invention is directed to an oral solid dosage form comprising a therapeutically effective amount of bupropion or a pharmaceutically acceptable salt thereof and one or more pharmaceutically acceptable excipients, the dosage form providing an in-vitro dissolution rate using USP Apparatus I (baskets) at 75 rpm in 1000 mL of 0.1N. HCl of not more than about 2% of bupropion or pharmaceutically acceptable salt thereof released after a time point selected from the group consisting of 2 hours, 4 hours, 8 hours, 12 hours, or 16 hours, the formulation providing a therapeutic effect such that the dosage form is administrable to a subject on a once-daily basis

In certain embodiments, the present invention is further directed to an oral solid dosage form comprising a therapeutically effective amount of bupropion or a pharmaceutically acceptable salt thereof and one or more pharmaceutically acceptable excipients, the dosage form providing an in-vitro dissolution rate when measured by USP <724> Method A, using USP Apparatus 2 (paddles) at 50 rpm in 0.1N. HCl for 2 hours, followed by a buffered solution, pH of 6.8 thereafter, which is less than about 5% bupropion or pharmaceutically acceptable salt thereof released after 2 hours; from about 20 to about 65% bupropion or pharmaceutically acceptable salt thereof released after 5 hours; from about 50% to about 90% bupropion or pharmaceutically acceptable salt thereof released after 8 hours; and not less than about 60% bupropion or pharmaceutically acceptable salt thereof released after 12 hours, the formulation providing a therapeutic effect such that the dosage form is administrable to a subject on a once-daily basis.

In certain embodiments, the present invention is further directed to an oral solid dosage form comprising a therapeutically effective amount of bupropion or a pharmaceutically acceptable salt thereof and one or more pharmaceutically acceptable excipients, the dosage form providing an in-vitro dissolution rate when measured by USP <724> Method A, using USP Apparatus 2 (paddles) at 50 rpm in 0.1N. HCl for 2 hours, followed by a buffered solution, pH of 6.8 thereafter, which is less than about 3% bupropion or pharmaceutically acceptable salt thereof released after 2 hours; from about 35 to about 55% bupropion or pharmaceutically acceptable salt thereof released after 5 hours; from about 60% to about 80% bupropion or pharmaceutically acceptable salt thereof released after 8 hours; and not less than about 70% bupropion or pharmaceutically acceptable salt thereof released after 12 hours, the formulation providing a therapeutic effect such that the dosage form is administrable to a subject on a once-daily basis.

In certain embodiments, the present invention is further directed to an oral solid dosage form comprising a therapeutically effective amount of bupropion or a pharmaceutically acceptable salt thereof and one or more pharmaceutically acceptable excipients, the dosage form providing an in-vitro dissolution rate using USP Apparatus I (baskets) at 75 rpm in 100 mL of water which is from about 3 to about 20% bupropion or pharmaceutically acceptable salt thereof released after 2 hours; from about 15% to about 45% bupropion or pharmaceutically acceptable salt thereof released after 4 hours; from about 45% to about 85% bupropion or pharmaceutically acceptable salt thereof released after 8 hours; and not less than about 60% bupropion or pharmaceutically acceptable salt thereof released after 12 hours, the formulation providing a therapeutic effect such that the dosage form is administrable to a subject on a once-daily basis.

In certain embodiments, the present invention is further directed to an oral solid dosage form comprising a therapeutically effective amount of bupropion or a pharmaceutically acceptable salt thereof and one or more pharmaceutically acceptable excipients, the dosage form providing an in-vitro dissolution rate using USP Apparatus I (baskets) at 75 rpm in 1000 mL of water which is from about 4 to about 15% bupropion or pharmaceutically acceptable salt thereof released after 2 hours; from about 20% to about 40% bupropion or pharmaceutically acceptable salt thereof released after 4 hours; from about 55% to about 75% bupropion or pharmaceutically acceptable salt thereof released after 8 hours; and not less than about 70% bupropion or pharmaceutically acceptable salt thereof released after 12 hours, the formulation providing a therapeutic effect such that the dosage form is administrable to a subject on a once-daily basis.

In certain embodiments, the present invention is further directed to an oral formulation for once-a-day administration of bupropion, comprising a therapeutically effective amount of bupropion or a pharmaceutically acceptable salt thereof, the bupropion or salt thereof being dispersed within a compressed tablet core comprising hydroxypropylcellulose and optional additional pharmaceutical excipients; a seal layer, and an enteric coating covering the seal layered tablet core.

In certain preferred embodiments, the amount of bupropion included in the formulation of the present invention is from about 75 to about 450 mg, and in most preferred embodiments, about 150 mg or about 300 mg, based on the hydrochloride salt.

In certain preferred embodiments, the formulation further comprises a seal coating covering the core and preferably disposed between the core and the enteric coating layer.

In certain embodiments, the extended release dosage form of the present invention provides a bio-curve equivalent to the bio-curve of Wellbutrin XL®. For example the dosage form of the present invention preferably provides a time to maximum plasma concentration (Tmax) of bupropion at from about 4 to about 6 hours after oral administration of the dosage form to a human patient and is bioequivalent to Wellbutrin XL®.

In certain embodiments, the present invention is further directed to a method of treating depression to a patient in need thereof, comprising administering a dosage form as disclosed herein to the patient.

In certain embodiments, the present invention is further directed to a method of preparing an oral solid dosage form of bupropion or a pharmaceutically acceptable salt thereof comprising forming a controlled release core comprising bupropion or a pharmaceutically acceptable salt thereof and a controlled release material; and coating the core with an enteric coating.

In certain embodiments, the method of preparation comprises forming a granulation comprising the bupropion or pharmaceutically acceptable salt thereof; blending the granulation with the controlled release material; compressing the blend into a tablet core with an optional pharmaceutically acceptable excipient; and coating the tablet core with an enteric coating.

In certain embodiments, the method of preparation comprises forming a granulation comprising the bupropion or pharmaceutically acceptable salt thereof; and the controlled release material; compressing the granulation into a tablet core with an optional pharmaceutically acceptable excipient; and coating the tablet core with an enteric coating.

The term “bioequivalent” is meant for purposes of the present invention to mean that the dosage form provides an AUC (bioavailability) and Cmax (rate of absorption) of from about 80% to about 125% of a reference standard, e.g., Wellbutrin XL.

The term “Tmax” is meant for purposes of the present invention as the mean time from administration of a dosage form to achieve the maximum concentration (Cmax) of the bupropion.

### DETAILED DESCRIPTION

The dosage forms of the present invention preferably provide for therapeutically effective treatment of depression (e.g., major depressive episode) in a human patient over a once-a-day (e.g., 24 hour) time period by providing for the controlled release of bupropion or a pharmaceutically acceptable salt thereof. Bupropion is an antidepressant of the aminoketone class, and is chemically unrelated to tricyclic, tetracyclic, selective serotonin re-uptake inhibitor, or other known antidepressant agents. Its structure closely resembles that of diethylpropion and it is related to phenylethylamines.

“Pharmaceutically acceptable salts” of bupropion, as used herein, is meant to encompass all pharmaceutically acceptable salts of bupropion, including, but not limited to, metal salts such as sodium salt, potassium salt, cesium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like; inorganic acid salts such as hydrochloride, hydrobromide, sulfate, phosphate and the like; organic acid salts such as formate, acetate, trifluoroacetate, maleate, fumarate, tartrate and the like; sulfonates such as methanesulfonate, benzenesulfonate, p-toluenesulfonate, and the like; amino acid salts such as arginate, asparginate, glutamate and the like. The preferred salt form of bupropion for use in accordance with the present invention is bupropion hydrochloride. Bupropion hydrochloride powder is white, crystalline, and highly soluble in water.

In a preferred embodiment, the dosage form of the present invention is directed to a controlled release solid oral dosage form comprising: (i) a core comprising a controlled release matrix comprising bupropion or pharmaceutically acceptable salt thereof dispersed in a controlled release material, and optionally one or more pharmaceutically acceptable excipients; and (ii) an enteric coating over the core; the dosage form providing a therapeutic effect for about 24 hours after administration to a human patient.

A non-limiting list of suitable controlled release materials for use in the core of the present invention includes hydrophilic and/or hydrophobic materials, such as sustained release polymer gums, acrylic resins, waxes, and oils such as hydrogenated castor oil, and hydrogenated vegetable oil. Suitable sustained-release polymers include alkylcelluloses such as ethylcellulose, acrylic and methacrylic acid polymers and copolymers (such as Eudragit® commercially available by Rohm Pharma); and cellulose ethers, such as hydroxyalkylcelluloses (e.g., hydroxypropylmethylcellulose) and carboxyalkylcelluloses. Examples of acrylic and methacrylic acid polymers and copolymers include methyl methacrylate, methyl methacrylate copolymers, ethoxyethyl methacrylates, ethyl acrylate, trimethyl ammonioethyl methacrylate, cyanoethyl methacrylate, aminoalkyl methacrylate copolymer, poly(acrylic acid), poly(methacrylic acid), methacrylic acid alkylamine copolymer, poly(methyl methacrylate), poly(methacrylic acid)(anhydride), polymethacrylate, polyacrylamide, poly(methacrylic acid anhydride), and glycidyl methacrylate copolymers. Waxes include for example natural and synthetic waxes, fatty acids, fatty alcohols, and mixtures of the same (e.g., beeswax, carnauba wax, stearic acid and stearyl alcohol). Certain embodiments utilize mixtures of any of the foregoing controlled release materials in the matrix of the core. However, any pharmaceutically acceptable hydrophobic or hydrophilic controlled release material which is capable of imparting controlled release of the bupropion or salt thereof may be used in accordance with the present invention.

Cellulosic polymers for use in the core of the present invention include hydroxyethyl cellulose hydroxypropylmethyl cellulose, hydroxypropylcellulose, sodium carboxy methyl cellulose, and mixtures thereof. A preferred extended release carrier is hydroxypropylcellulose (“HPC”). Examples of HPC polymers are available from Hercules Incorporation, Aqualon Division under the tradename KLUCEL®.

An example of a class of polymers that may be further included in the core of the present invention in addition to the controlled release material is polyvinylpyrrolidone (also known as povidone or PVP). The United States Pharmacopoeia XXII describes povidone as a synthetic polymer consisting essentially of linear 1-vinyl-2-pyrrolidone groups. The polyvinylpyrrolidones, are commonly available from BASF under the brand name Kollidon, or from ISP under the brand name Plasdone®. The grade of Kollidon® that is selected depends mainly on its molecular weight, as this dictates the viscosity, binding effect, the complexation capacity and how readily it is eliminated from the body. Kollidon® 25, 30 and 90 F give hard, free-flowing granules with a low proportion of fines and high binding strength when compressed into tablets. For Kollidon® 25, and 30, the quantity can be from about 2% to about 5% of the tablet weight. For Kollidon® 90 F, less than about 2% can be utilized because of its great binding capacity. The high viscosity of binder solutions made with Kollidon® 90 F sometimes requires certain precautions to ensure that the granules are evenly wetted. Granulators or spraying machines that have a strong mixing action are helpful. Polyvinylpyrrolidone is available as a water soluble polymer. Examples of water soluble polyvinylpyrrolidones, include PVP K-12, PVP K-15, PVP K-17, PVP K-25, PVP K-30, PVP K-60, PVP K-90, and PVP K-120 having approximate molecular weights of 2500, 8000, 10000, 30000, 50000, 400000, 1000000, and 3000000, respectively. Soluble PVP is conventionally used as a binder in tablet formulations.

Another example of a class of polymers that may be used in the present invention are water soluble vinylpyrrolidone-vinyl acetate copolymers that are formed by the copolymerization of vinylpyrrolidone and vinyl acetate. An example of a vinylpyrrolidone-vinyl acetate copolymer that may be used in the present invention is the copolymer available from BASF under the brand name Kollidon® VA-64.

Another example of a class of polymers that may be used in the present invention are carbomers. Carbomers are synthetic high-molecular-weight polymers of acrylic acid that are cross-linked with either allylsucrose or allyl ethers of pentaerythritol. Carbomers are typically used as dry or wet binders and as a rate controlling excipient. Certain carbomers for use in certain embodiments of the present invention include for example, Carbopol® 941, 971 PNF, 981 and 71G manufactured by Noveon, Inc.

In certain preferred embodiments, the vinylpyrrolidone-vinyl acetate copolymer and carbomer (e.g., carbopol) are used in combination.

In accordance with certain embodiments of the present invention, stabilizers may be included, such as for example, organic acids, carboxylic acids, acid salts of amino acids, and sodium metabisulphite. The acid salts of amino acids can be hydrochloride salts such as cysteine hydrochloride, glycine hydrochloride or cystine dihydrochloride. Other examples of stabilizers which can be utilized in the formulations disclosed herein include: ascorbic acid, malic acid, isoascorbic acid, citric acid, tartaric acid, L-cysteine hydrochloride, and glycine hydrochloride. In preferred embodiments, the bupropion formulations provide a suitable degradation profile upon accelerated storage conditions without the necessity to utilize any of the stabilizers described above.

In preferred embodiments, the dosage forms of the present invention do not utilize a stabilizer and are prepared as stable formulations by utilizing a drying step or steps during manufacture wherein the moisture content of the tablet core is reduced to less than about 2.0%, preferably to about 1.5%.

In certain embodiments, the bupropion is wet granulated with one or more excipients and the granulation is then dried to a target moisture content of less than about 2%, less than about 1.5% or less than about 1%, preferably to about 0.7%. The dried granulation is then optionally combined with other excipients (which can be subject to a separate drying step before being combined with the granulation) and compressed into tablets. The tablets can then be dried to a moisture content of less than about 2%, preferably to about 1.5%. A separate drying step to achieve a moisture content of less than about 2%, preferably to about 1.5%, can also be performed after the seal coat and/or after the enteric coat. In certain embodiments, the process for stabilizing the dosage form without a stabilizer utililizes at least one of the drying steps described above.

In addition to the above ingredients, a controlled release formulation of the present invention may also contain suitable quantities of other materials, e.g. diluents, lubricants, flow-aids, binders, pore-formers, granulating aids (e.g., solvents and acidifying agents), colorants, flavorants and glidants that are conventional in the pharmaceutical art. The quantities of these additional materials will be sufficient to provide the desired effect to the desired formulation. Specific examples of pharmaceutically acceptable carriers and excipients that may be used to formulate oral dosage forms are described in the Handbook of Pharmaceutical Excipients, American Pharmaceutical Association (1986), incorporated by reference herein.

Certain examples of lubricants include stearic acid, magnesium stearate, carnauba wax, glyceryl behenate, talc, mineral oil (in PEG), mixtures thereof, and the like. Magnesium stearate and carnauba wax are preferred lubricants.

Certain examples of binders include water-soluble polymer, such as modified starch, gelatin, polyvinylpyrrolidone, polyvinyl alcohol, povidone, sodium carboxymethylcellulose, alginic acid, poly(ethylene glycol), poly(propylene glycol), guar gum, polysaccharide, bentonite clay, sugar, poloxamer, collagen, albumin, gelatin, mixtures thereof, and the like.

Certain examples of fillers or diluents for use in the present invention include for example, lactose, microcrystalline cellulose, dextrin, dextrose, starch, mixtures thereof and the like.

Certain examples of glidants for use in the present invention include for example, calcium phosphate tribasic, calcium silicate, powdered cellulose, colloidal silicon dioxide, magnesium silicate, magnesium trisilicate, silicon dioxide, starch, talc, mixtures thereof and the like.

Certain examples of a flow-aid include silicone products such as colloidal silicone dioxide.

The relative amounts of ingredients in the core are preferably as follows. The proportion of bupropion hydrochloride in the core may be, e.g., from about 40% to about 80% of the core dry weight. The proportion of lubricant in the core may be, e.g., from about 0.1 to about 10% of the core dry weight. The proportion of binder or filler in the core may be, e.g., from about 5% to about 40% of the core dry weight.

In accordance with the invention, the core may further be coated with a seal coating. In a preferred embodiment, the seal coating occurs between the core and the enteric coating. The seal coating may comprise a hydrophilic polymer. Examples include but are not limited to hydroxypropylcellulose, hydroxypropylmethylcellulose, methoxypropyl cellulose, hydroxypropylisopropylcellulose, hydroxypropylpentylcellulose, hydroxypropylhexylcellulose and any mixtures thereof. The seal coat may also be a polymer such as polyvinyl alcohol.

The seal coating may be applied by press coating, molding, spraying, dipping and/or air-suspension or air tumbling procedures. A preferred method of applying the seal coating is by pan coating, where the seal coating is applied by spraying it onto the cores accompanied by tumbling in a rotating pan. The seal coating material may be applied to the cores as a suspension by employing solvents, e.g., an organic, aqueous, or a mixture of an organic and aqueous solvent. Exemplary solvents suitable in applying the seal coating include aqueous-based solutions, an alcohol, ketone, ester, ether, aliphatic hydrocarbon, halogenated solvents, cycloaliphatic solvents, aromatic, heterocyclic, aqueous solvents, and mixtures thereof. In a preferred embodiment, the seal coating comprises hydroxypropyl cellulose and hydroxypropylmethylcellulose, and is delivered as a suspension using a suitable solvent such as e.g., ethanol or water.

The core of the present invention is preferably coated with an enteric layer. The enteric materials for use in the enteric layer preferably resist the action of gastric fluid preferably allowing for release of the active agent in the intestinal tract.

Suitable enteric coating may comprise cellulose acetate phthalate, polyvinyl acetate phthalate, acrylic resins such as Eudragit L®., shellac, cellulose acetate butyrate, hydroxypropyl methylcellulose phthalate or combinations thereof.

Additional materials suitable for use in the enteric coating include phthalates including cellulose acetyl phthalate, cellulose triacetyl phthalate, cellulose acetate tetrahydrophthalate, cellulose acetate trimellilate, sodium cellulose acetate phthalate, cellulose ester phthalate, cellulose ether phthalate, methylcellulose phthalate, cellulose ester-ether phthalate, hydroxy propyl cellulose phthalate, alkali salts of cellulose acetate phthalate, alkaline earth salts of cellulose acetate phthalate, calcium salt of cellulose acetate phthalate, ammonium salt of hydroxypropyl methylcellulose phthalate, cellulose acetate hexahydrophthalate, hydroxypropyl methylcellulose hexahydrophthalate, and polyvinyl acetate phthalate, may be used as gastroresistant, enterosoluble coatings for the formulations of the present invention. Other enteric coatings may be used as long as they do not readily dissolve or disperse in the gastric juices of the stomach but do dissolve or disperse in the intestinal fluid of the intestines. An acrylic-based film-coating system, commercially available as Acryl-EZE® by Colorcon, West Point, Va., is a preferred enteric coating. Enteric materials are also particularly preferred because they form an impermeable barrier which will not readily dissolve or disperse at the low pH provided by the gastric juices in the stomach. The enteric materials are discussed in Remington's Pharmaceutical Sciences, 17th Ed., page 1637 (1985).

The enteric coating may be applied by press coating, molding, spraying, dipping and/or air-suspension or air tumbling procedures. One method of applying the enteric coating is by pan coating, where the enteric coating is applied by spraying the enteric composition onto the cores accompanied by tumbling in a rotating pan. The enteric coating material may be applied to the cores by employing solvents, including an organic, aqueous or a mixture of an organic and aqueous solvent. Exemplary solvents suitable in applying the enteric coating include an alcohol, ketone, ester, ether, aliphatic hydrocarbon, halogenated solvents, cycloaliphatic solvents, aromatic, heterocyclic, aqueous solvents, and mixtures thereof.

In certain embodiments, the enteric coating can have an organic or inorganic pore-former included in the coating that can be dissolved, extracted or leached from the enteric coating in the environment of use. For example, the pore-formers may comprise one or more water-soluble hydrophilic polymers in order to modify the release characteristics of the formulation. Examples of suitable hydrophilic polymers include hydroxypropylmethylcellulose, cellulose ethers, acrylic resins, lactose and protein-derived materials. Of these polymers, the cellulose ethers, especially hydroxyalkylcelluloses and carboxyalkylcelluloses, are preferred. Also, synthetic water-soluble polymers may be used, such as polyvinylpyrrolidone, polyethylene glycol, polyethylene oxide, etc., and polysaccharides, e.g., pullulan, dextran, etc.

Examples of forms of preferred solid pharmaceutical composition include a tablet or capsule. Preferably the pharmaceutical formulation of the present invention is a tablet. Such forms are prepared using standard procedures known in the art which involve admixing bupropion or a pharmaceutically acceptable salt thereof, the controlled release material with other optional excipients and compressing the mixture into a core. The core is coated with an optional seal layer and then with an enteric layer as disclosed above. Optionally the core may be inserted into a capsule and the capsule coated with the enteric layer.

In preferred embodiments the formulations of the present invention includes the following ingredients:

| | |
|---|---|
| active agent | bupropion hydrochloride |
| binder | copovidone (e.g., Kollidone ® VA 64) |
| sustained release polymer | hydroxypropylcellulose (e.g., |
| | Klucel ® HXF) |
| flow-aid | colloidal silicone dioxide (e.g., |
| | Cab-o-sil ® M-5P) |
| lubricant | magnesium stearate |
| protecting coating material | Opadry II |
| enteric coating material | methacrylic acid copolymer type C (e.g., |
| | Acryl-EZE ®) |
| pore-forming agent | povidone (e.g., Plasdone ® K 29/32) |
| solvent | purified water |

### DETAILED DESCRIPTION OF THE PREFERRED EMBODIMENTS

The following examples illustrate various aspects of the present invention. They are not to be construed to limit the claims in any manner whatsoever.

### EXAMPLE 1

A bupropion HCl matrix tablet core was prepared having the formulation in Table 1:

TABLE 1

| Ingredients | Percent (%) |
|---|---|
| Bupropion HCl | 60.00 |
| Klucel HXF | 30.75 |
| Kollidon VA 64 | 8.00 |
| SiO₂ | 0.5 |
| Magnesium Stearate | 0.75 |

Based on the percentage of ingredients in Table 1, tablet cores containing 150 mg bupropion HCL was prepared having the following ingredients listed in Table 2 below:

TABLE 2

| Ingredient | Amount needed (mg) |
|---|---|
| Bupropion HCI | 150 |
| Klucel HXF | 76.88 |
| Kollidon VA 64 | 20 |
| SiO₂ | 1.25 |
| Magnesium Stearate | 1.88 |

A “seal” coat was prepared and applied to the matrix tablet cores disclosed above. The seal coat was prepared having the formulation in Table 3:

TABLE 3

| Ingredients | Mg per Tablet |
|---|---|
| Opadry ® II, white | 5 |

An enteric coating was then prepared and applied to the seal coated matrix tablet cores. The enteric coat was prepared having the formulation of Table 4:

TABLE 4

| Ingredient | Amount (Mg) |
|---|---|
| Acryl-EZE ® | 23.74 |
| Plasdone ® K-29/32 | 1.26 |

The tablets of Example 1 were prepared using the following process:

Bupropion Hydrochloride Granulation—Fluid Bed Process

1. Charge the Bupropion HCl and Kollidon VA 64 into a 45 L bowl of a Fluid Bed Processor.

2. Activate the Fluid Bed Processor and adjust the settings to achieve a product temperature of approximately 30° C.

3. Begin spraying the a binder solution of Kollidon VA 64 at a rate of 50 g/min.±10 g/min.

4. Continue spraying operations while adjusting parameters to achieve a product temperature between 22 and 35° C., inclusive, with a spray rate of no more than 150 g/min.

5. After the Binder Solution is depleted rinse the solution container with Purified Water and spray the rinse solution onto the product bed at a rate between 50 and 80 g/min.

6. Adjust process parameters until the product temperature reaches approximately 44° C. Using a moisture balance determine the moisture content of the granulation. Continue drying the product until a representative sample demonstrates a moisture content of <0.7%.

7. When the drying process is completed pulse the filters on the Fluid Bed Processor to remove any residual product. Discharge all the product from processor bowl.

Bupropion Hydrochloride Granulation—Milling Process

1. Slowly charge the dried Bupropion HCl granulation into a Quadro Co-Mil (or equivalent) which has been fitted with the appropriate impeller, spacer disc and 0.039 inches/18 mesh screen.

2. Adjust the settings to obtain a speed of approximately 2000 rpm and collect the milled material.

Bupropion Hydrochloride ER Tablet Blend—Klucel Drying Process

1. Dry a sufficient amount of Klucel HXF on trays in a Forced Air Oven Dryer with an inlet air temperature of 80° C. ±5° C. Dry the material for a minimum of two hours.

2. Using a moisture balance determine the moisture content of the Klucel. Continue drying the Klucel until a representative sample demonstrates a moisture content of NMT 1.3%.

Bupropion Hydrochloride ER Tablet Blend—Blending Process

1. Weigh the requisite amounts of Bupropion HCl Granulation, Cab-O-Sil M-5P, and Magnesium Stearate NF into properly labeled containers.

2. Charge the Bupropion HCl Granulation into a 5 cubic foot PK Blender.

3. Mix the Cab-O-Sil M-5P and approximately half of the dried Klucel HXF from the previous process in a suitable container. Pass this mixture through a 20 mesh pan screen. Charge this material into the blender.

4. Screen the remaining portion of Klucel HXF through the 20 mesh screen and add it into the blender.

5. Blend materials for 10 minutes.

6. Screen the Magnesium Stearate through a 30 mesh pan screen and add to the contents of the blender.

7. Blend the materials in the blender for 5 minutes and collect the material.

Bupropion Hydrochloride ER Tablet Cores—Compression Process

1. Weigh the requisite amount of tablet blend into appropriately labeled containers.

2. Set the parameters for the processing room and tablet press (Korsch XL-100 or equivalent) to obtain tablet cores with the following specifications:

| | |
|---|---|
| Weight: Target - 250 mg | Average - 242.5 mg to 257.5 mg; |
| | Individual - 225 mg to 275 mg |
| Thickness: Target - 5.1 mm | Range - 4.6 mm to 5.6 mm |
| Hardness: Target - 9.5 kp | Average - 7.5 kp to 11.5 kp; |
| | Individual - 6.5 kp to 12.5 kp |

Friability: Conforms to USP <1216> Tablet Friability

3. Charge the blend into the tablet press hopper.

4. Begin compression of the blend and collect the tablet cores.

Bupropion Hydrochloride ER Tablets—Seal Coating Solution Preparation

1. Agitate Purified Water with a propeller mixer, creating a vortex.

2. Slowly add in the Opadry II and mix for 45 minutes.

3. While performing the mixing step adjust the pH of the Seal Coating Solution to 2.0±0.5 with concentrated Hydrochloric acid.

Bupropion Hydrochloride ER Tablets—Enteric Coating Solution Process

1. Agitate Purified Water with a propeller mixer, creating a vortex.

2. Slowly add in the Plasdone K-29/32 and mix for 15 minutes.

3. Add in the Acryl-EZE to the solution and mix for an additional 50 minutes.

4. Pass the Enteric Coating Solution through a 60 mesh hand screen into an appropriate container.

Bupropion Hydrochloride ER Tablets—Pump RPM Setting for Enteric Coat Process

1. Using a small portion of Seal Coating Solution determine the RPM setting necessary to achieve a spray rate of 70 g/min. ±5 g/min.

2. Verify that the RPM setting is correct.

3. After verification, clear the pump line of solution and rinse with purified water.

Bupropion Hydrochloride ER Tablets—Pump RPM Setting for Enteric Coat Process

1. Using a small portion of Seal Coating Solution determine the RPM setting necessary to achieve a spray rate of 60 g/min. ±5 g/min.

Bupropion Hydrochloride ER Tablets—Solution Coating Process

1. Charge the tablet cores into the coating pan of a pan coater.

2. Adjust the pan coater settings to achieve a product temperature of 44° C.±10° C.

3. Once the product reaches the specified temperature determine the moisture content of the cores by analyzing a sample using a moisture balance. Continue heating the product until a representative sample demonstrates a moisture content of less than 1.3%.

4. When the specified moisture level is achieved determine the average weight of the tablet cores using a 50 tablet sample.

5. Adjust the average weight determined above by factoring in an additional 5.0 mg to represent the theoretical weight of the seal coat. Use this calculated weight (±1 mg) as the target weight for the tablets at the end of the seal coat process.

6. Agitate the Seal Coating Solution with the propeller mixer. Continue mixing the solution throughout the coating process.

7. Connect the solution line to the Seal Coating solution. Set the pump to the rpm value determined above for the Seal Coating Process.

8. Begin spraying process. Adjust the settings for the pan coater parameters to maintain the following process conditions:
Product temperature of 44° C.±10° C.
Inlet air temperature of 60° C.±15° C.
Pan speed of 10 rpm±5 rpm
Inlet air flow at 900 cfm±120 cfm

9. Continue the spraying process until the target weight determined in step 7 above for the coated tablets is achieved.

10. When the target weight is achieved stop the spray function of the pan coater. Maintain the product temperature at 44° C.±10° C. and dry the coated tablets for 10 minutes.

11. Determine the moisture content of the coated tablets using the moisture balance. If the moisture content is more than 1.3% continue to dry the tablets and check the moisture content of the tablets every 10 minutes until the target moisture level is obtained.

12. Clear the Seal Coating Solution from the solution line then rinse the line with purified water.

13. Connect the solution line to the Enteric Coating Solution. Purge the line with Enteric Coating Solution.

14. Determine the average weight of the coated tablets using a 50 tablet sample.

15. Adjust the average weight determined above by factoring in an additional 25 mg to represent the theoretical weight of the enteric coating. Use this calculated weight (±2.5 mg) as the target weight for the tablets at the end of the enteric coating process.

16. Set the pump to the rpm value determined above for the Enteric Coating Process.

17. Begin spraying process. Adjust the settings for the pan coater parameters to maintain the following process conditions:
Product temperature of 44° C.±10° C.
Inlet air temperature of 60° C.±15° C.
Pan speed of 10 rpm±5 rpm
Inlet air flow at 900 cfm±120 cfm

18. Continue the spraying process until the target weight determined in step 15 above for the enteric coated tablets is achieved.

19. When the target weight is achieved stop the spray function of the pan coater and maintain the pan speed at 10 rpm±5 rpm and set the pan to jog position. Allow the product to dry for 30 minutes.

20. Collect all acceptable tablets in appropriate containers

### EXAMPLE 2

A 300 mg bupropion HCl matrix tablet core was prepared in accordance with Example 1, having the same formulation in Table 1. The 300 mg buproprion HCl matrix tablet core of Example 2 is dose proportional to Example 1; thus, based on the percentage of ingredients in Table 1, the formulation of Example 2 is as found in Table 5 below:

TABLE 5

| Ingredient | Amount needed (mg) |
|---|---|
| Bupropion HCI | 300 |
| Klucel HXF | 153.76 |
| Kollidon VA 64 | 40 |
| SiO₂ | 2.5 |
| Magnesium Stearate | 3.76 |

A “seal” coat was prepared and applied to the matrix tablet cores disclosed above. The seal coat was prepared having the formulation in Table 6:

TABLE 6

| Ingredients | Mg per Tablet |
|---|---|
| Opadry ® II, white | 10 |

An enteric coating was then prepared and applied to the seal coated matrix tablet cores. The enteric coat was prepared having the formulation of Table 7:

TABLE 7

| Ingredient | Amount (Mg) |
|---|---|
| Acryl-EZE ® | 47.5 |
| Plasdone ® K-29/32 | 2.52 |

The tablets of Example 2 were prepared using the process disclosed in Example 1.

### EXAMPLE 3

Bupropion HCl tablets prepared according to Example 1, and Wellbutrin XL 150 mg tablets were tested for in vitro dissolution based upon USP <724> Method A, using USP Apparatus 2 (paddles) at 50 rpm in 1000 mL of 0.1N. HCl for 2 hours, followed by buffered solution, pH of 6.8 thereafter. The following results were obtained for each formulation:

TABLE 8

| | Example 1 | | | | | |
|---|---|---|---|---|---|---|
| Vessel # | 2 hours | 3 hours | 5 hours | 8 hours | 12 hours | 16 hours |
| 1 | 0.8 | 18.5 | 46.3 | 71.8 | 82.8 | 83.2 |
| 2 | 0.8 | 17.9 | 43.2 | 69.5 | 82.6 | 83.3 |
| 3 | 1.6 | 18.7 | 48.1 | 72.7 | 82.5 | 83.1 |
| 4 | 0.8 | 20.1 | 46.7 | 71.8 | 83.1 | 83.7 |
| 5 | 0.8 | 20.1 | 48.7 | 74.4 | 84.6 | 85.1 |
| 6 | 0.8 | 19.7 | 48.6 | 72.3 | 83.8 | 84.3 |
| 7 | 0.7 | 18.0 | 44.4 | 72.6 | 85.3 | 85.6 |
| 8 | 0.7 | 19.1 | 46.8 | 74.9 | 85.2 | 85.6 |
| 9 | 0.6 | 18.4 | 43.7 | 70.9 | 81.9 | 82.7 |
| 10 | 0.6 | 16.6 | 44.4 | 71.8 | 84.8 | 85.3 |
| 11 | 0.7 | 16.2 | 41.2 | 68.9 | 84.0 | 84.5 |
| 12 | 0.6 | 19.3 | 44.4 | 70.6 | 81.8 | 82.4 |
| Mean | 1 | 19 | 46 | 72 | 84 | 84 |
| SD | 0.3 | 1.2 | 2.4 | 1.8 | 1.3 | 1.2 |
| Min | 1 | 16 | 41 | 69 | 82 | 82 |
| Max | 2 | 20 | 49 | 75 | 85 | 86 |
| % RSD | 37.9 | 6.5 | 5.3 | 2.5 | 1.6 | 1.4 |

TABLE 9

| | Wellbutrin XL | | | | | |
|---|---|---|---|---|---|---|
| Vessel # | 2 hours | 3 hours | 5 hours | 8 hours | 12 hours | 16 hours |
| 1 | 1.9 | 27.4 | 58.1 | 83.1 | 92.8 | 95.4 |
| 2 | 1.6 | 25.7 | 57.2 | 82.1 | 92.9 | 96.0 |
| 3 | 1.6 | 26.0 | 58.2 | 87.2 | 94.7 | 96.4 |
| 4 | 2.3 | 32.6 | 66.8 | 91.1 | 96.2 | 97.9 |
| 5 | 2.8 | 33.4 | 64.8 | 88.0 | 93.7 | 95.7 |
| 6 | 2.1 | 29.3 | 61.2 | 86.4 | 95.2 | 97.4 |
| 7 | 2.4 | 29.8 | 59.4 | 86.6 | 94.1 | 96.6 |
| 8 | 1.7 | 26.5 | 59.4 | 88.3 | 95.6 | 97.9 |
| 9 | 3.3 | 33.3 | 65.5 | 90.5 | 96.6 | 98.7 |
| 10 | 1.8 | 27.3 | 59.9 | 85.2 | 93.5 | 96.2 |
| 11 | 1.5 | 24.9 | 57.2 | 84.2 | 93.8 | 96.4 |
| 12 | 1.5 | 27.6 | 60.2 | 86.8 | 93.7 | 95.2 |
| Mean | 2 | 29 | 61 | 87 | 94 | 97 |
| SD | 0.6 | 3.0 | 3.3 | 2.7 | 1.3 | 1.0 |
| Min | 2 | 25 | 57 | 82 | 93 | 95 |
| Max | 3 | 33 | 67 | 91 | 97 | 99 |
| % RSD | 30.0 | 10.3 | 5.4 | 3.1 | 1.4 | 1.0 |

### EXAMPLE 4

Bupropion HCl, 150 mg tablets prepared in accordance with Example 1 and Wellbutrin XL 150 mg tablets were tested for in vitro dissolution using USP Apparatus I (baskets) at 75 rpm in 1000 mL of 0.1N. HCl. The following results were obtained:

TABLE 10

| | Example 1 | | | | |
|---|---|---|---|---|---|
| Vessel # | 2 hours | 4 hours | 8 hours | 12 hours | 16 hours |
| 1 | 0.6 | 0.3 | 0.4 | 0.4 | 0.7 |
| 2 | 0.7 | 0.3 | 0.3 | 0.7 | 3.6 |
| 3 | 0.6 | 0.4 | 0.4 | 0.4 | 0.6 |
| 4 | 0.7 | 0.4 | 0.5 | 0.8 | 3.1 |
| 5 | 1.7 | 0.3 | 0.3 | 0.4 | 0.5 |
| 6 | 0.0 | 0.0 | 0.0 | 1.7 | 0.3 |
| Mean | 1 | 0 | 0 | 1 | 1 |
| SD | 0.5 | 0.0 | 0.2 | 0.5 | 1.5 |
| Min | 0 | 0 | 0 | 0 | 0 |
| Max | 2 | 0 | 1 | 2 | 4 |
| % RSD | 50.0 | 52.0 | 45.1 | 50.0 | 150.0 |

TABLE 11

| | Wellbutrin XL 150 mg | | | | |
|---|---|---|---|---|---|
| Vessel # | 2 hours | 4 hours | 8 hours | 12 hours | 16 hours |
| 1 | 3.0 | 33.6 | 83.2 | 94.1 | 96.3 |
| 2 | 2.6 | 24.3 | 86.1 | 98.3 | 99.6 |
| 3 | 2.4 | 27.7 | 75.4 | 93.4 | 96.8 |
| 4 | 2.0 | 26.6 | 74.0 | 94.0 | 97.2 |
| 5 | 2.0 | 26.7 | 76.3 | 93.6 | 96.6 |
| 6 | 2.2 | 27.3 | 81.9 | 94.5 | 97.1 |
| Mean | 2 | 28 | 79 | 95 | 97 |
| SD | 0.4 | 3.1 | 4.9 | 1.8 | 1.2 |
| Min | 2 | 24 | 74 | 93 | 96 |
| Max | 3 | 34 | 86 | 98 | 100 |
| % RSD | 20.0 | 11.1 | 6.2 | 1.9 | 1.2 |

### EXAMPLE 5

Bupropion HCl, 150 mg tablets prepared in accordance with Example 1 and Wellbutrin XL 150 mg tablets were tested for in vitro dissolution using USP Apparatus I (baskets) at 75 rpm in 1000 mL of water. The following results were obtained:

TABLE 12

| | Example 1 | | | | |
|---|---|---|---|---|---|
| Vessel # | 2 hours | 4 hours | 8 hours | 12 hours | 16 hours |
| 1 | 4.3 | 26.0 | 63.8 | 84.5 | 90.8 |
| 2 | 8.7 | 32.8 | 69.5 | 88.0 | 91.5 |
| 3 | 3.9 | 26.7 | 66.4 | 87.4 | 92.3 |
| 4 | 4.1 | 24.0 | 61.1 | 83.1 | 90.1 |
| 5 | 4.4 | 28.9 | 68.0 | 87.8 | 93.0 |
| 6 | 6.5 | 28.7 | 65.6 | 85.1 | 91.0 |
| 7 | 7.2 | 31.5 | 68.2 | 87.0 | 91.1 |
| 8 | 6.1 | 29.3 | 67.9 | 88.5 | 93.0 |
| 9 | 5.1 | 27.8 | 66.1 | 86.8 | 91.9 |
| 10 | 7.7 | 31.9 | 69.6 | 89.3 | 94.4 |
| 11 | 6.0 | 30.1 | 66.1 | 86.0 | 91.1 |
| 12 | 4.7 | 26.4 | 65.1 | 85.7 | 91.6 |
| Mean | 6 | 29 | 66 | 87 | 92 |
| SD | 1.6 | 2.6 | 2.4 | 1.8 | 1.2 |
| Min | 4 | 24 | 61 | 83 | 90 |
| Max | 9 | 33 | 70 | 89 | 94 |
| % RSD | 26.7 | 9.0 | 3.6 | 2.1 | 1.3 |

TABLE 13

| | Wellbutrin XL 150 mg | | | | |
|---|---|---|---|---|---|
| Vessel # | 2 hours | 4 hours | 8 hours | 12 hours | 16 hours |
| 1 | 0.9 | 7.9 | 33.2 | 56.1 | 75.4 |
| 2 | 0.6 | 7.4 | 27.8 | 48.3 | 68.6 |
| 3 | 0.6 | 5.3 | 23.7 | 40.1 | 56.9 |
| 4 | 0.8 | 9.4 | 28.9 | 48.9 | 66.7 |
| 5 | 0.8 | 7.8 | 32.1 | 55.2 | 74.2 |
| 6 | 0.9 | 9.9 | 30.9 | 52.2 | 72.1 |
| 7 | 0.8 | 6.1 | 31.6 | 56.6 | 74.9 |
| 8 | 0.8 | 10.4 | 28.0 | 44.9 | 61.9 |
| 9 | 0.8 | 3.1 | 28.3 | 48.6 | 67.5 |
| 10 | 0.6 | 2.7 | 28.4 | 50.4 | 69.3 |
| 11 | 0.9 | 9.4 | 34.0 | 58.3 | 78.1 |
| 12 | 0.9 | 9.4 | 31.3 | 52.8 | 72.9 |
| Mean | 1 | 7 | 30 | 51 | 70 |
| SD | 0.1 | 2.6 | 2.9 | 5.3 | 6.1 |
| Min | 1 | 3 | 24 | 40 | 57 |
| Max | 1 | 10 | 34 | 58 | 78 |
| % RSD | 10.0 | 37.1 | 9.7 | 10.4 | 8.7 |

What is claimed is:

1. An oral solid dosage form comprising a controlled release core comprising bupropion or a pharmaceutically acceptable salt thereof and a controlled release material; said core overcoated with an enteric coating.

2. The oral solid dosage form of claim 1, wherein said dosage form provides a therapeutic effect such that the formulation is suitable for once-a-day administration to a human subject.

3. The oral solid dosage form of claim 1, wherein said core comprises a controlled release matrix comprising said bupropion or pharmaceutically acceptable salt thereof dispersed in said controlled release material.

4. The oral solid dosage form of claim 3, wherein said controlled release matrix is a compressed tablet.

5. The oral solid dosage form of claim 1, wherein said enteric coating is selected from the group consisting of cellulose acetate phthalate, polyvinyl acetate phthalate, acrylic resins, shellac, cellulose acetate butyrate, hydroxypropyl methylcellulose phthalate or combinations thereof.

6. The oral solid dosage form of claim 1, further comprising a seal coating.

7. The oral solid dosage form of claim 6, wherein said seal coating is disposed between the controlled release core and the enteric coating.

8. The oral solid dosage form of claim 6, wherein said seal coating is selected from the group consisting of hydroxypropylcellulose, hydroxypropylmethylcellulose, methoxypropylcellulose, hydroxypropylisopropylcellulose, hydroxypropylpentylcellulose, hydroxypropylhexylcellulose and mixtures thereof.

9. The oral solid dosage form of claim 1, wherein said controlled release material comprises a hydrophilic polymer.

10. The oral solid dosage form of claim 9, wherein said hydrophilic polymer comprises hydroxypropylcellulose.

11. The oral solid dosage form of claim 1, wherein said controlled release material comprises a hydrophobic polymer.

12. The oral solid dosage form of claim 11, wherein said hydrophobic polymer comprises ethylcelllulose.

13. The oral solid dosage form of claim 1, which provides an in-vitro dissolution rate using USP Apparatus I (baskets) at 75 rpm in 1000 mL of 0.1N. HCl of not more than about 10% of bupropion or pharmaceutically acceptable salt thereof released after 8 hours.

14. The oral solid dosage form of claim 1, which provides an in-vitro dissolution rate using USP Apparatus I (baskets) at 75 rpm in 1000 mL of 0.1N. HCl of not more than about 5% of bupropion or pharmaceutically acceptable salt thereof released after 8 hours.

15. The oral solid dosage form of claim 1, wherein the enteric coating has a weight gain from about 5% to about 15% of the final dosage form.

16. The oral solid dosage form of claim 1, which provides an in-vitro dissolution rate when measured by USP <724> Method A, using USP Apparatus 2 (paddles) at 50 rpm in 1000 mL 0.1N. HCl for 2 hours, followed by a buffered solution, pH of 6.8 thereafter, which is less than about 5% bupropion or pharmaceutically acceptable salt thereof released after 2 hours; from about 20 to about 65% bupropion or pharmaceutically acceptable salt thereof released after 5 hours; from about 50% to about 90% bupropion or pharmaceutically acceptable salt thereof released after 8 hours; and not less than about 60% bupropion or pharmaceutically acceptable salt thereof released after 12 hours.

17. The oral solid dosage form of claim 1, which provides an in-vitro dissolution rate when measured by USP <724> Method A, using USP Apparatus 2 (paddles) at 50 rpm in 1000 mL of 0.1N. HCl for 2 hours, followed by a buffered solution, pH of 6.8 thereafter, which is less than about 3% bupropion or pharmaceutically acceptable salt thereof released after 2 hours; from about 35 to about 55% bupropion or pharmaceutically acceptable salt thereof released after 5 hours; from about 60% to about 80% bupropion or pharmaceutically acceptable salt thereof released after 8 hours; and not less than about 70% bupropion or pharmaceutically acceptable salt thereof released after 12 hours.

18. An oral solid dosage form comprising a therapeutically effective amount of bupropion or a pharmaceutically acceptable salt thereof and one or more pharmaceutically acceptable excipients, said dosage form providing an in-vitro dissolution rate using USP Apparatus I (baskets) at 75 rpm in 1000 mL of 0.1N. HCl of not more than about 10% of bupropion or pharmaceutically acceptable salt thereof released after 2 hours, said formulation providing a therapeutic effect such that the dosage form is administrable to a subject on a once-daily basis.

19. An oral solid dosage form comprising a therapeutically effective amount of bupropion or a pharmaceutically acceptable salt thereof and one or more pharmaceutically acceptable excipients, said dosage form providing an in-vitro dissolution rate using USP Apparatus I (baskets) at 75 rpm in 1000 mL of 0.1N. HCl of not more than about 5% of bupropion or pharmaceutically acceptable salt thereof released after 2 hours, said formulation providing a therapeutic effect such that the dosage form is administrable to a subject on a once-daily basis.

20. An oral solid dosage form comprising a therapeutically effective amount of bupropion or a pharmaceutically acceptable salt thereof and one or more pharmaceutically acceptable excipients, said dosage form providing an in-vitro dissolution rate using USP Apparatus I (baskets) at 75 rpm in 1000 mL of water which is from about 3 to about 20% bupropion or pharmaceutically acceptable salt thereof released after 2 hours; from about 15% to about 45% bupropion or pharmaceutically acceptable salt thereof released after 4 hours; from about 45% to about 85% bupropion or pharmaceutically acceptable salt thereof released after 8 hours; and not less than about 60% bupropion or pharmaceutically acceptable salt thereof released after 12 hours, the formulation providing a therapeutic effect such that the dosage form is administrable to a subject on a once-daily basis.

21. An oral solid dosage form comprising a therapeutically effective amount of bupropion or a pharmaceutically acceptable salt thereof and one or more pharmaceutically acceptable excipients, said dosage form providing an in-vitro dissolution rate using USP Apparatus I (baskets) at 75 rpm in 1000 mL of water which is from about 4 to about 15% bupropion or pharmaceutically acceptable salt thereof released after 2 hours; from about 20% to about 40% bupropion or pharmaceutically acceptable salt thereof released after 4 hours; from about 55% to about 75% bupropion or pharmaceutically acceptable salt thereof released after 8 hours; and not less than about 70% bupropion or pharmaceutically acceptable salt thereof released after 12 hours, the formulation providing a therapeutic effect such that the dosage form is administrable to a subject on a once-daily basis.

22. A method of treating depression comprising administering to a subject in need thereof an effective amount of a pharmaceutical composition according to claim 1.

23. The method of claim 23, wherein said administration is on a once-a-day basis.

24. A method of preparing an oral solid dosage form of bupropion or a pharmaceutically acceptable salt thereof comprising:
(a) forming a controlled release core comprising bupropion or a pharmaceutically acceptable salt thereof and a controlled release material; and
(b) coating the core with an enteric coating.

25. The method of claim 24, wherein the controlled release core is formed by:
(a) forming a granulation comprising said bupropion or pharmaceutically acceptable salt thereof;
(b) blending said granulation with the controlled release material;
(c) compressing the blend into a tablet core with an optional pharmaceutically acceptable excipient; and
(d) coating said tablet core with an enteric coating.

26. The method of claim 25, wherein the controlled release core is formed by:
(a) forming a granulation comprising said bupropion or pharmaceutically acceptable salt thereof; and the controlled release material;
(b) compressing the granulation into a tablet core with an optional pharmaceutically acceptable excipient; and
(c) coating said tablet core with an enteric coating.

27. The method of claim 24, further comprising drying said core to a moisture content of less than about 2%, preferably to 1.5%, prior to step (b).

27. The method of claim 25, further comprising drying said granulation to a moisture content of less than about 2%, preferably to about 0.7%.

28. The method of claim 25, further comprising drying said core to a moisture content of less than about 2%, preferably to about 1.5%, prior to step (c).

29. The method of claim 25, further comprising drying said controlled release material to a moisture content of less than about 2%, preferably to about 1.5%, prior to step (b).
